SEQUENCE LISTING (38 sequences)

SEQ ID NO: 1 - protein, 12 aa, Artificial Sequence
  Synthetic
LLADTTHHRPWT

SEQ ID NO: 2 - protein, 12 aa, Artificial Sequence
  Synthetic
GQVLPTTTPSSP

SEQ ID NO: 3 - protein, 12 aa, Artificial Sequence
  Synthetic
VPQHPYPVPSHK

SEQ ID NO: 4 - protein, 12 aa, Artificial Sequence
  Synthetic
HNMAPATLHPLP

SEQ ID NO: 5 - protein, 12 aa, Artificial Sequence
  Synthetic
QSFASLTNPRVL

SEQ ID NO: 6 - protein, 12 aa, Artificial Sequence
  Synthetic
HTTPTTTYAAPP

SEQ ID NO: 7 - protein, 12 aa, Artificial Sequence
  Synthetic
QYGVVSHLTHTP

SEQ ID NO: 8 - protein, 12 aa, Artificial Sequence
  Synthetic
TMSNPITSLISV

SEQ ID NO: 9 - protein, 12 aa, Artificial Sequence
  Synthetic
IGRISTHAPLHP

SEQ ID NO: 10 - protein, 12 aa, Artificial Sequence
  Synthetic
MNDPSPWLRSPR

SEQ ID NO: 11 - protein, 12 aa, Artificial Sequence
  Synthetic
QSLGSMFQEGHR

SEQ ID NO: 12 - protein, 12 aa, Artificial Sequence
  Synthetic
KPLFTRYGDVAI

SEQ ID NO: 13 - protein, 12 aa, Artificial Sequence
  Synthetic
MPFGARILSLPN

SEQ ID NO: 14 - protein, 12 aa, Artificial Sequence
  Synthetic
QLQLSNSMSSLS

SEQ ID NO: 15 - protein, 12 aa, Artificial Sequence
  Synthetic
TMNMPAKIFAAM

SEQ ID NO: 16 - protein, 12 aa, Artificial Sequence
  Synthetic
EPTKEYTTSYHR

SEQ ID NO: 17 - protein, 12 aa, Artificial sequence
  Synthetic
DLNELYLRSLRA

SEQ ID NO: 18 - protein, 12 aa, Artificial Sequence
  Synthetic
DYDSTHGAVFRL

SEQ ID NO: 19 - protein, 12 aa, Artificial Sequence
  Synthetic
SKHERYPQSPEM

SEQ ID NO: 20 - protein, 12 aa, Artificial Sequence
  Synthetic
HTHSSDGSLLGN

SEQ ID NO: 21 - protein, 12 aa, Artificial Sequence
  Synthetic
NYDSMSEPRSHG

SEQ ID NO: 22 - protein, 12 aa, Artificial Sequence
  Synthetic
ANPIISVQTAMD

SEQ ID NO: 23 - protein, 12 aa, Artificial Sequence
  Description of Artificial Sequence Synthetic peptide
VIGESTHHRPWS

SEQ ID NO: 24 - protein, 12 aa, Artificial Sequence
  Description of Artificial Sequence Synthetic peptide
IIGESSHHKPFT

SEQ ID NO: 25 - protein, 12 aa, Artificial Sequence
  Description of Artificial Sequence Synthetic peptide
GLGDTTHHRPWG

SEQ ID NO: 26 - protein, 12 aa, Artificial Sequence
  Description of Artificial Sequence Synthetic peptide
ILAESTHHKPWT

SEQ ID NO: 27 - protein, 60 aa, Artificial Sequence
  Description of Artificial Sequence Synthetic polypeptide
LLADTTHHRPWTVIGESTHHRPWSIIGESSHHKPFTGLGDTTHHRPWGILAESTHHKPWT

SEQ ID NO: 28 - protein, 48 aa, Artificial Sequence
  Description of Artificial Sequence Synthetic polypeptide
LLADTTHHRPWTVIGESTHHRPWSIIGESSHHKPFTGLGDTTHHRPWG

SEQ ID NO: 29 - protein, 36 aa, Artificial Sequence
  Description of Artificial Sequence Synthetic polypeptide
LLADTTHHRPWTVIGESTHHRPWSIIGESSHHKPFT

SEQ ID NO: 30 - protein, 24 aa, Artificial Sequence
  Description of Artificial Sequence Synthetic peptide
LLADTTHHRPWTVIGESTHHRPWS

SEQ ID NO: 31 - protein, 48 aa, Artificial Sequence
  Description of Artificial Sequence Synthetic polypeptide
VIGESTHHRPWSIIGESSHHKPFTGLGDTTHHRPWGILAESTHHKPWT

SEQ ID NO: 32 - protein, 114 aa, Homo sapiens
QAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 33 - protein, 217 aa, Artificial Sequence
  Description of Artificial Sequence Synthetic polypeptide
MPIGSLLADTTHHRPWTVIGESTHHRPWSIIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 34 - protein, 212 aa, Artificial Sequence
  Description of Artificial Sequence Synthetic polypeptide
LLADTTHHRPWTVIGESTHHRPWSIIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 35 - protein, 200 aa, Artificial Sequence
  Description of Artificial Sequence Synthetic polypeptide
VIGESTHHRPWSIIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 36 - protein, 188 aa, Artificial Sequence
  Description of Artificial Sequence Synthetic polypeptide
IIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 37 - protein, 176 aa, Artificial Sequence
  Description of Artificial Sequence Synthetic polypeptide
GLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 38 - protein, 164 aa, Artificial Sequence
  Description of Artificial Sequence Synthetic polypeptide
ILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

(19) United States
(12) Patent Application Publication
(10) Pub. No.: US 2023/0233740 A1
(43) Pub. Date: Jul. 27, 2023

(54) OSTEOINDUCTIVE BONE REGENERATION MATERIAL AND PRODUCTION METHOD OF THE SAME

(71) Applicants: ORTHOREBIRTH CO., LTD., Yokohama-shi (JP); THERADAPTIVE, INC., Frederick, MD (US)

(72) Inventors: Luis ALVAREZ, Lexington, MA (US); Yasutoshi NISHIKAWA, Yokohama-shi (JP); Hiroyuki TAIRA, Naha-shi (JP)

(21) Appl. No.: 18/002,608

(22) PCT Filed: Jun. 18, 2021
(86) PCT No.: PCT/JP2021/023279

§ 371 Date: Dec. 20, 2022

Related U.S. Application Data
(60) Provisional application No. 63/042,006, filed on Jun. 21, 2020.

(51) Int. Cl. A61L 27/46

(52) U.S. Cl. CPC A61L 27/46; A61L 2430/02

(57) ABSTRACT

A method of producing an osteoinductive bone graft formed of a plurality of electrospun biodegradable fibers is disclosed. The method includes preparing a fibrous scaffold material formed of the plurality of electrospun biodegradable fibers, wherein the plurality of electrospun biodegradable fibers are entangled with each other to form a cotton-wool like structure having inter-fiber spaces forming a microenvironment for cell growth therein, and immersing the fibrous scaffold in a solution containing BMP-2 so that the BMP-2 is bound to the calcium particles exposed on the surface of the fibers. Area of binding site for BMP-2 on calcium particles exposed on a surface of the electrospun biodegradable fibers is adjusted by an amount of the calcium particles contained in the electrospun biodegradable fibers.

### TECHNICAL FIELD

The present invention relates to osteoinductive bone regeneration materials and production methods for the osteoinductive bone regeneration materials.

### BACKGROUND ART

One of the Applicants has been producing and selling an osteoconductive bone regeneration material formed of biodegradable fibers containing (β-TCP under a tradename ReBOSSIS. The bone regeneration material is produced by using an electrospinning process, in which a spinning solution is ejected as a thin fiber from a nozzle and pulled by the electrostatic attraction in the electric field to be deposited on a collector. Using a novel electrospinning setup, the Applicant have successfully prepared such biodegradable fibers into a cotton wool-like structure, which contains 13-TCP and a biodegradable polymer. The cotton wool-like structure is unique and confers several advantages: (1) it contains a large interstitial space to allow biological fluids to readily permeate into the bone graft structure, (2) it offers a large surface area to allow ready release of calcium and phosphorus from (β-TCP into the biological fluids; (3) it has a flexible structure that can be made to conform to the shape of the bone repair site; and (4) it offers a large surface area for cells to attach. In vivo and in vitro evaluation of the cotton wool-like composite as bone substitute material has demonstrated its advantages in the repair of complex bone defects.

Bone morphogenetic protein-2 (BMP-2) is osteoinductive and can facilitates bone formation/regeneration. For example, Infuse™ Bone Graft (Medtronic) contains a man-ufactured bone graft material containing recombinant human BMP-2 (rhBMP-2) and is approved by the Food and Drug Administration (FDA) for use as a bone graft in sinus augmentation and localized alveolar ridge augmentation. BMP-2 is incorporated into a bone implant (INFUSE) and delivered to the site of the fracture. BMP-2 is gradually released at the site to stimulate bone formation; the growth stimulation by BMPs is localized and sustained for some weeks. If BMP-2 leaks into remote sites, adverse effects would occur. Indeed, several side effects arising from rhBMP-2 have been reported. These side effects include postoperative inflammation and associated adverse effects, ectopic bone formation, osteoclast-mediated bone resorption, and inappropriate adipogenesis.

Therefore, there is a need for bone graft materials that can include BMP-2 in a manner that allows for gradual release of the BMP-2 to achieve bone formation in an intended site but would not permit leaching of this growth factor to an unintended site.

### SUMMARY OF INVENTION

In order to solve the problems of osteoinductive bone graft in the prior art, inventors of the present invention made intensive studies and found that (β-TCP particles exposed on the surfaces of the biodegradable fibers of ReBOSSIS are not covered by a thin polymer layer. Based on that finding, inventors of the present invention concluded that the exposed portion of therβ-TCP particles can be used to bind BMP-2 to the particles. By binding BMP-2 to the β-TCP particles, one can use ReBOSSIS as a scaffold for an osteoinductive bone regeneration material that allows gradual release of BMP-2 in the bone defect site but would not permit leaching of this growth factor to an unintended site.

Embodiments of the present invention relate to osteoinductive bone regeneration materials that comprise ReBOSSIS fibers and a bone morphogenetic protein-2 (BMP-2). The BMP for use with embodiments of the invention may be a BMP-2 or a derivative of BMP-2. The BMP-2 may be a human BMP-2 or an animal (e.g., pets or livestock) BMP-2. A derivative of BMP-2 includes a BMP-2 fused with one or more 13-TCP binding peptides to form a fusion protein, which will be referred to in this description as a “targetable BMP-2” or “tBMP-2.” All these different forms of BMP-2, such as human BMP-2 (including recombinant human BMP-2, rhBMP-2 and wild-type human BMP-2, wtBMP-2), animal BMP-2, and tBMP-2, may be referred to generically as “BMP-2.” That is, the term “BMP-2” encompasses rhBMP-2, wtBMP-2, animal BMP-2, and tBMP-2.

ReBOSSIS has a cotton-wool like structure formed of a plurality of electrospun biodegradable fibers having a diameter of 40-320 μm, length of 5-20 mm and containing calcium compound particles (e.g., β-TCP particles) and biodegradable polymer such as poly(lactic acid) (PLLA) or poly(lactic-co-glycolic acid) (PLGA). The biodegradable fibers may contain other calcium compound particles, such as silicon releasing calcium carbonate vaterite (i.e., silicon-doped vaterite, SiV). Thus, ReBOSSIS fibers may comprise biodegradable polymer (e.g., PLLA and/or PLGA) and calcium compound particles (e.g., β-TCP particles and/or SiV particles). As used herein, the term “calcium compound particles” may be β-TCP particles, SiV particles, or a combination of 13-TCP particles and SiV particles.

An electrospun biodegradable fiber of ReBOSSIS contains a large amount of calcium compound particles distributed on or in the fibers. A portion of the β-TCP particles are exposed on the surface of the fibers forming uneven surface topography of the fiber, and the remaining portion of the β-TCP particles are buried in the fibers. The β-TCP particles exposed on the surface of the fiber is not coated by a thin polymer layer. By immersing ReBOSSIS in a solution containing bone morphogenic protein 2 (BMP-2, including tBMP-2), BMP-2 is bound to the β-TCP particles and/or SiV particles exposed on the surface of the fibers so that the BMP-2 is captured on the β-TCP particles and/or SiV particles exposed on the surface of the fibers of ReBOSSIS throughout the cotton-wool like structure.

Uneven surface topography of the biodegradable fiber of ReBOSSIS helps stem cells to attach to the fibers. Area of binding site for BMP-2 on β-TCP particles exposed on a surface of the electrospun biodegradable fibers may be increased or decreased by increasing or decreasing the amount of the β-TCP particles contained in the electrospun biodegradable fibers.

The biodegradable fibers have diameter of about 40-320 μm, preferably about 70-250 μm, more preferably 90-200 μm such that calcium compound particles (e.g., β-TCP and/or SiV particles) having a diameter of about 2-5 μm can be distributed in the fiber and mechanical strength of the cotton wool-like structure can be maintained after implantation of ReBOSSIS at the site of a bone defect.

In an embodiment of the present invention, lengths of the biodegradable fibers are about 5 to 20 mm, more preferably about 4 to 10 mm. Because ReBOSSIS is formed of such short fibers entangled each other, the cotton wool-like structure can be easily separated into smaller pieces by hand. Therefore, a surgeon can make a cotton wool-like material in accordance with the size of bone defect of a patient by separating the desired smaller size from ReBOSSIS without using a cutter or scissor.

Preferably, diameters of the electrospun biodegradable fibers are adapted such that the fibrous scaffold maintains a sufficient mechanical strength and an average size of a channel of the fibrous scaffold is in a range of 10-300 μm after the fibrous scaffold is implanted in a bone defect site. As used herein, a channel of the fibrous scaffold refers to a passage formed by inter-fiber spaces in the fibrous scaffold.

After implantation of ReBOSSIS at a bone defect site, body fluids containing mesenchymal stem cells may come into contact with the BMP-2 (e.g., rhBMP-2 or t-BMP-2) captured on the β-TCP particles. Then, the BMP-2 (e.g., rhBMP-2 or tBMP-2) promotes osteoprogenitor cells to differentiate into osteoblast cells. The β-TCP particles that bind BMP-2 may be gradually dissolved by osteoclast cells. Then, the osteoblast cells work to form bone on the β-TCP particles (i.e., bone remodeling).

After implantation of ReBOSSIS at a bone defect site, biodegradable polymer (e.g., PLLA and/or PLGA) in the electrospun fibers are gradually degraded such that the β-TCP particles buried in the fibers gradually become exposed, and the newly exposed 13-TCP particles may recapture the BMP-2 (e.g., rhBMP-2 or tBMP-2) that were adhered on the surface of the fibers. As the degradation of polymer proceeds, due to the recapture of BMP-2 (e.g., rhBMP-2 or tBMP-2) by the newly exposed β-TCP particles, remodeling of bone continuously occurs throughout the network of the scaffold of biodegradable fibers, resulting in efficient bone formation at the bone defect site.

Due to the binding of BMP-2 (e.g., rhBMP-2 or tBMP-2) to the β-TCP particles that are fixed to a surface of biodegradable fiber, the BMP-2 is prevented from leaking to outside of bone defect area. As a result, safety of using the BMP-2/ReBOSSIS is ensured.

In one aspect, provided herein is a composition comprising: a scaffold comprising about 60 wt % to about 80 wt % calcium containing compound, and a targetable BMP-2 comprising (i) VIGESTHHRPWS (SEQ ID NO: 23), (ii) IIGESSHHKPFT (SEQ ID NO: 24), (iii) GLGDTTHHRPWG (SEQ ID NO: 25), (iv) ILAESTHHKPWT (SEQ ID NO: 26), or (v) a combination of two more of (i)-(iv). In some embodiments, the targetable BMP-2 comprises VIGESTHHRPWS (SEQ ID NO: 23). In some embodiments, the targetable BMP-2 comprises IIGESSHHKPFT (SEQ ID NO: 24). In some embodiments, the targetable BMP-2 comprises GLGDTTHHRPWG (SEQ ID NO: 25). In some embodiments, the targetable BMP-2 comprises ILAESTHHKPWT (SEQ ID NO: 26). In some embodiments, the targetable BMP-2 further comprises LLADTTHHRPWT (SEQ ID NO: 1). In some embodiments, the targetable BMP-2 comprises QAKHKQRKRLKSSCKRHPLYVDFSDVGWNDWIVAPPGYHAFY-CHGECPFPLADHLNSTNHAIVQTLVNSVNSKIPKACCVPTELSAISMLYLDENE KVVLKNYQDMVVEGCGCR (SEQ ID NO: 32). In some embodiments, the targetable BMP-2 comprises any one of SEQ ID NOS: 33-38. In some embodiments, the targetable BMP-2 comprises SEQ ID NO: 33. In some embodiments, the calcium containing compound comprises calcium phosphate, vaterite, or calcium phosphate and vaterite. In some embodiments, the calcium containing compound comprises beta-tricalcium phosphate (β-TCP). In some embodiments, the β-TCP is present in the scaffold at about 60 wt % to about 80 wt % of the scaffold. In some embodiments, the β-TCP is present in the scaffold at about 70 wt %. In some embodiments, the β-TCP is present in the scaffold at about 30 wt % to about 50 wt % of the scaffold. In some embodiments, the β-TCP is present in the scaffold at about 40 wt %. In some embodiments, the calcium containing compound comprises vaterite. In some embodiments, the vaterite is present in the scaffold at about 20 wt % to 40 wt % of the scaffold. In some embodiments, the vaterite is present in the scaffold at about 30 wt %. In some embodiments, the vaterite comprises SiV (silicon-doped vaterite). In some embodiments, the scaffold comprises a biodegradable polymer. In some embodiments, the scaffold comprises poly(lactic-co-glycolic acid) (PLGA). In some embodiments, the scaffold comprises about 20 wt % to about 40 wt % PLGA. In some embodiments, the scaffold comprises about 30 wt % PLGA. Further provided are methods of treating a subject with a composition and/or structure provided herein. For example, to treat a bone defect in the subject.

### BRIEF DESCRIPTION OF DRAWINGS

FIGS. 1A-1F show electron microscope images of ReBOSSIS fibers. FIG. 1A shows the image of several ReBOSSIS(85) fibers (PLGA 30 wt %, SiV 30 wt %, β-TCP 40 wt %) at 200× magnification, showing interstitial spaces between fibers in the cotton wool-like structure. FIG. 1B shows the image of one ReBOSSIS(85) fiber at 2000× magnification. The calcium particles on the surface of the fiber are readily discernable.

FIG. 1C shows the same fiber at 5000× magnification, in which the white arrows indicate the β-TCP particles and the dark arrows indicate the SiV particles. FIG. 1D shows the image of several fibers (PLGA 30 wt %, β-TCP 70 wt %) at 200×magnification. FIG. 1E shows the image of a fiber (PLGA 30 wt %, β-TCP 70 wt %) at 2000× magnification. FIG. 1F shows the same fiber (PLGA 30 wt %,β-TCP 70 wt %) at 5000× magnification, in which the white arrows indicate the β-TCP particles.

FIG. 2 shows an SDS-PAGE gel image illustrating the binding of tBMP-2 having SEQ ID NO: 33 to ReBOSSIS (85) as compared with a control (BSA). Panel A shows a gel image obtained using an acidic buffer (acetate buffer) for wash buffer, and Panel B shows a gel image obtained using a neutral buffer (PBS) for wash buffer. In each gel image, the four right lanes show results of analysis of tBMP-2, and the four left lanes show results of analysis of BSA.

FIG. 3 shows a gel image from the binding of tBMP-2 having SEQ ID NO: 33 to several calcium containing materials. tBMP2 binds well to the materials (SiV70, ReBOSSIS (85), and ORB-03) containing 13-TCP and/or SiV. Lane 1 is a marker, lane 2 is empty, lanes 3-6 show the amount of BSA that bound to PLGA (lane 3), SiV70 (lane 4), ReBOSSIS(85) (lane 5), or ORB-03 (lane 6) after an acetate-low pH wash, lane 7 is empty, lanes 8-11 show the amount of tBMP2 that bound to PLGA (lane 8), SiV70 (lane 9), ReBOSSIS(85) (lane 10), or ORB-03 (lane 11) after an acetate-low pH wash, and lane 12 is empty. FIG. 10 describes the procedure for the binding assay.

FIG. 4 shows a gel image from the binding of rhBMP-2 (recombinant human BMP-2, not linked to a β-TCP binding peptide) to several calcium containing materials. rhBMP-2 is primarily retained on materials containing β-TCP (ReBOSSIS (85) and ORB-03), but not on material containing SiV only. Lane 1 is a marker, lane 2 is empty, lanes 3-6 show the amount of BSA that bound to PLGA (lane 3), SiV70 (lane 4), ReBOSSIS(85) (lane 5), or ORB-03 (lane 6) after an acetate-low pH wash, lane 7 is empty, lanes 8-11 show the amount of rhBMP-2 that bound to PLGA (lane 8), SiV70 (lane 9), ReBOSSIS(85) (lane 10), or ORB-03 (lane 11) after an acetate-low pH wash, and lane 12 is empty. FIG. 10 describes the procedure for the binding assay.

FIG. 5 shows a schematic of Chronic Caprine Critical Defect (CCTD) Model. A 5-cm segment of critical defect is created in skeletally mature female goats during the pre-procedure. A 5-cm long ×2 cm diameter polymethylmethacrylate (PMMA) spacer is placed in the defect to induce a biological membrane. Four weeks later, the PMMA spacer is gently removed and replaced with the grafting materials. Orthogonal radiographs are taken every four weeks to assess defect healing. In the figure, AP represents craniocaudal, and ML represents mediolateral. White arrows indicate grafting material in placement of PMMA spacers.

FIGS. 6A-6B show the radiographs (mediolateral (ML) and craniocaudal (AP) projections) taken 8 weeks (FIG. 6A) and 12 weeks (FIG. 6B) after grafting surgery. 6 goats per treatment group. The tBMP-2-containing groups having SEQ IS NO: 33 (Group 2 (0.15 mg/cc tBMP2) and Group 3 (1.5 mg/cc tBMP2)) showed higher percentages of new bone formation compared to Group 1 (without tBMP-2). Radiographs from Group 1 are shown in the first two columns, radiographs from Group 2 are shown in the second two columns, and radiographs from Group 3 are shown in the last two columns of the figure.

FIG. 7 shows radiographs (mediolateral (ML) and craniocaudal (AP) projections) of the 12 explanted tibias taken with a fixed x-ray machine. Large amount of new bone was obtained in the higher dose tBMP-2 group (1.5 mg/cc, Group 3). The addition of tBMP-2 to TCP and ReBOSSIS enhanced the bone healing in the CCTD model. Radiographs from Group 1 are shown in the first two columns, radiographs from Group 2 are shown in the second two columns, and radiographs from Group 3 are shown in the last two columns of the figure.

FIG. 8 is a conceptual diagram of percolation phenomenon, illustrating the formation of β-TCP particle clusters when the amounts of β-TCP particles exceed the percolation threshold.

FIG. 9A shows surface of electrospun PLGA fiber that contains β-TCP particles of 50 wt % (24.3 vol %). FIG. 9 B shows surface of electrospun PLGA fiber that contains β-TCP particles of 70 wt % (42.9 vol %). FIG. 9C shows surface of electrospun PLGA fiber that contains β-TCP particles of 80 wt % (56.3 vol %). FIG. 9D shows surface of electrospun PLGA fiber that contains β-TCP particles of 85 wt % (64.6 vol %).

FIG. 10 shows a diagram that explains a method of collecting samples for SDS-PAGE analysis.

FIG. 11 shows a conceptual diagram that explains a mechanism of bone regeneration according to an embodiment of the present invention.

FIG. 12 shows a conceptual diagram that explains a mechanism of bone regeneration according to an embodiment of the present invention.

FIGS. 13A-13D show experimental results evidencing that β-TCP particles exposed on a surface of electrospun biodegradable fiber containing 70 wt % β-TCP particles are not coated by a polymer.

FIGS. 14A-14E show experimental results evidencing that β-TCP particles exposed on a surface of electrospun biodegradable fiber containing 50 wt % β-TCP particles are not coated by a polymer.

### DESCRIPTION OF EMBODIMENTS

Embodiments of the invention relate to osteoinductive bone regeneration materials that contain calcium particles (β-TCP and/or SiV) and a bone morphogenetic protein-2 (BMP-2, such as rhBMP-2 or tBMP-2). In addition, the bone regeneration materials of the invention have a cotton wool-like structure such that the BMP-2, which is bound to a large surface area on the cotton wool-like structure, can interact with the biological fluids at the bone repair sites such that the osteoinduction process is facilitated.

Osteoinduction involves stimulation of osteoprogenitor cells to differentiate into osteoblasts that then begin new bone formation. In contrast, osteoconduction occurs when the bone graft material serves as a scaffold for new bone growth that is per-petuated by existing osteoblasts from the margin of the native bone surrounding the defect site.

Embodiments of the invention may use a recombinant BMP-2 (e.g., rhBMP-2) or a targetable BMP-2. A targetable BMP-2 is a BMP protein fused with alβ-TCP-binding peptide (i.e., a fusion protein) such that BMP-2 can bind tightly to β-TCP in the bone regeneration materials. The β-TCP binding peptide may be fused to the N- or C-terminus of the BMP-2.

BMP-2 have strong bone formation activities and are used in orthopedic applications, such as spinal fusion. However, BMP-2 may induce bone formation at the unintended sites, if they escape from the treatment sites. These BMP-2-associated complications occurred with relative high frequencies, ranging from 20% to 70% of cases, and these adverse effects could be potentially life threatening. (Aaron W. James et al., “A review of the Clinical Side Effects of Bone Morphogenetic Protein-2,” Tissue Eng. Part B Rev., 2016, 22(4): 284-297). Thus, it is essential that one confine the BMPs at the treatment sites, e.g., by securely binding BMP-2 to the bone regeneration/repair materials and not allow BMP-2 to diffuse away from the treatment sites.

Embodiments of the invention may use rhBMP-2 or BMP-2 fusion proteins that each contain one or more β-TCP binding peptides. These BMP-2 fusion proteins are referred to as targetable BMP-2 or tBMP-2. The tBMP-2 is designed to be used with 13-TCP containing and/or SiV containing bone regeneration/repair materials, in which the tBMP-2 bind tightly with β-TCP and/or SiV and would not diffuse away from the treatment sites, thereby eliminating or minimizing adverse effects.

The bone regeneration/repair materials of the invention have a cotton wool-like structure made of biodegradable fibers that comprise β-TCP and a biodegradable polymer (e.g., poly(lactic-co-glycolic acid; PLGA). The tBMP-2 fusion proteins can bind tightly to β-TCP and/or SiV particles on these cotton wool-like structures and would not diffuse away from the treatment sites.

The cotton wool-like structure confers several advantages: (1) it contains a large interstitial space to allow biological fluids to readily permeate into the bone graft structure, (2) it offers a large surface area to allow ready release of calcium and phosphorus from β-TCP into the biological fluids; (3) it offers a large surface area to support/carry other bioactive or bone morphogenetic factors, such as rhBMP-2 or tBMP-2; and (4) it has a flexible structure that can be made to conform to the shape of the bone repair site.

The cotton wool-like structures are produced by electrospinning a solution containing a biodegradable polymer and β-TCP. Details of the formation of the cotton wool-like structures are described in U.S. Pat. Nos. 8,853,298, and 10,092,650, U.S. patent application publication Nos. 2016/0121024, and 2018/0280569, the description of which are incorporated by reference in their entirety. These cotton wool-like materials are available from Orthorebirth Co., Ltd. (Yokohama, Japan) under the tradename ReBOSSIS.

ReBOSSIS has a cotton-wool like structure formed of a plurality of electrospun biodegradable fibers containing β-TCP and/or SiV particles and biodegradable polymer, such as poly(lactic-co-glycolic acid) (PLGA) or poly(lactic acid) (PLLA). The biodegradable fibers may contain β-TCP or other calcium compound particles, such as silicon releasing calcium carbonate (vaterite) (SiV). Silicon-doped vaterite (SiV) particles have been found to have the ability to enhance cell activities in biodegradable composite materials. (Obata et al., “Enhanced in vitro cell activity on silicon-doped vaterite/poly(lactic acid) composites,” Acta Biomater., 2009, 5(1): 57-62; doi: 10.1016/j.actbio.2008.08.004).

In ReBOSSIS, an electrospun biodegradable fiber contains a large amount of calcium compound particles (β-TCP and/or SiV particles) distributed in the fiber. In typical ReBOSSIS fibers, the calcium compound particles (e.g., β-TCP particles or β-TCP+SiV particles) can account for about 30-85 wt %, preferably about 50-80 wt %, and more preferably about 70-80 wt %. Containing such large amount of calcium particles in the biodegradable fiber is made possible by using kneading process. If the amount of calcium compound particles exceeds 85 wt %, it becomes difficult to knead the mixture of PLGA and calcium compound particles to disperse the particles in the polymer.

The calcium compound particles are denser than the PLGA. For example, the PLGA has a density of 1.01 g/cm3, and β-TCP has a density of 3.14 g/cm3. Thus, the wt % and vol % may have a correlation as follows:

TABLE 1
β-TCP content correlation

| | wt % | | | | | | | | |
|---|---|---|---|---|---|---|---|---|---|
| | 90 | 80 | 70 | 60 | 50 | 40 | 30 | 20 | 10 |
| vol % | 74.3 | 56.3 | 42.9 | 32.5 | 24.3 | 17.7 | 12.1 | 7.4 | 3.5 |

In some embodiments, provided are scaffolds or fibers comprising about 60 wt % to about 80 wt % calcium compound containing compounds. Non-limiting examples include calcium phosphate (e.g., beta-tricalcium phosphate (β-TCP)) and vaterite (e.g., silicon-doped vaterite (SiV)). In some cases, the scaffolds or fibers comprise about 70 wt % calcium compound containing compounds. As an example, the scaffolds or fibers comprise about 70 wt % β-TCP. As another example, the scaffolds or fibers comprise about 40 wt % β-TCP. As yet another example, the scaffolds or fibers comprise about 40% β-TCP and about 30% SiV. In accordance with embodiments of the invention, the contents of the ReBOSSIS(85) fibers may be referred to either in wt % or in vol %. For example, some ReBOSSIS(85) fibers may contain β-TCP in an amount of about 25-65 vol % and the PLGA in an amount of about 75-35 vol %, more preferably β-TCP particles 40-60 vol % and PLGA 60-40 vol %.

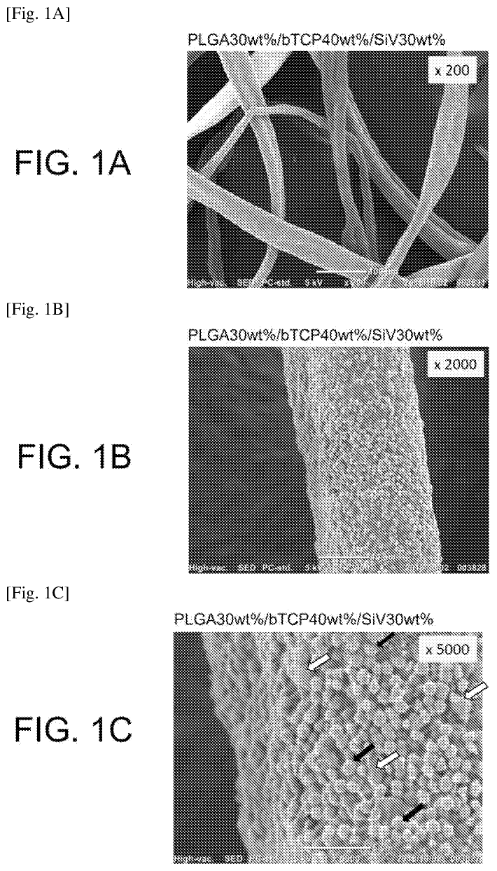

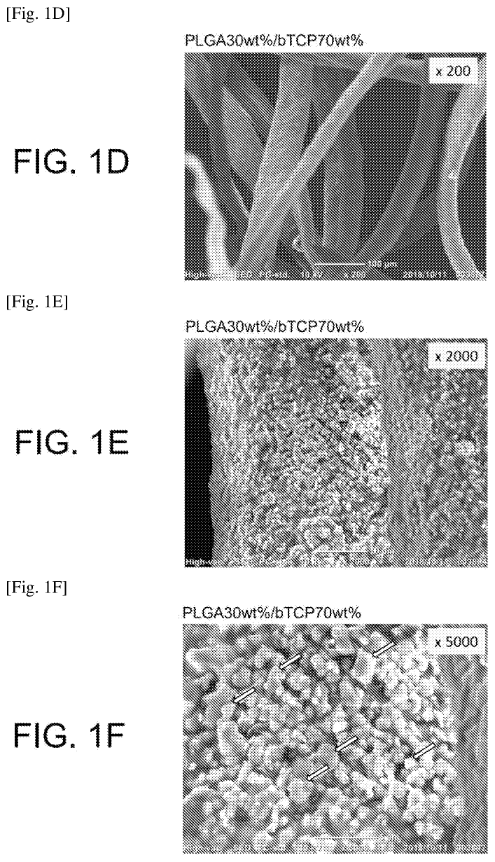

In accordance with embodiments of the invention, the scaffolds and fibers herein have a portion of the calcium compound particles (e.g., β-TCP particles, or SiV particles, or β-TCP+SiV particles) exposed on the surface of the fibers, while the remaining portion of calcium compound particles are buried inside the fibers. For example, FIGS. 1A-1F show scanning electron micrographs of two ReBOSSIS samples: ReBOSSIS(85) comprises PLGA (30 wt % or 50.8 vol %), SiV (30 wt % or 27.4 vol %), and β-TCP (40 wt % or 21.8 vol %), and ORB-03 comprises PLGA (30 wt % or 57.1 vol %) and β-TCP (70 wt % or 42.9 vol %).

FIG. 1A shows the image of several ReBOSSIS(85) fibers (PLGA 30 wt %, SiV 30 wt %, β-TCP 40 wt %) at 200× magnification, showing interstitial spaces between fibers in the cotton wool-like structure. The large interstitial volume between the fibers facilitates the perfusion of biological fluids. FIG. 1B shows the image of one ReBOSSIS(85) fiber at 2000× magnification. The calcium particles on the surface of the fiber are readily discernable. FIG. 1C shows the same fiber at 5000× magnification, in which the white arrows indicate the β-TCP particles and the dark arrows indicate the SiV particles. The large number of calcium particles exposed on the surfaces of the fibers provide sites for binding by the BMP-2 or tBMP-2. In addition, the exposed calcium particles also facilitate interactions with osteoclasts and osteoblasts during remodeling and new bone formation.

FIG. 1D shows the image of several ORB-03 fibers (PLGA 30 wt %/β-TCP 70 wt %) at 200× magnification, showing interstitial spaces between fibers in the cotton wool-like structure. The large interstitial volume between the fibers facilitates the perfusion of biological fluids. FIG. 1E shows the image of one ORB-03 fiber at 2000× magnification. The calcium particles on the surface of the fiber are readily discernable. FIG. 1F shows the same fiber at 5000× magnification, in which the white arrows indicate the β-TCP particles. The large number of calcium particles exposed on the surfaces of the fibers provide sites for binding by the BMP-2 or tBMP-2. In addition, the exposed calcium particles also facilitate interactions with osteoclasts and osteoblasts during remodeling and new bone formation.

In accordance with embodiments of the invention, scaffolds and fibers herein (e.g., ReBOSSIS(85), ORB-03) preferably have diameters from about 40 μm to about 320 μm (including any number in the range), preferably from about 70 μm to about 250 μm, more preferably from about 90 μm to about 200 μm, such that calcium compound particles having a diameter of 1-5 μm can be distributed in and on the fiber and the mechanical strength of the cotton wool-like structure is sufficient to maintain the desired shape after implantation of the scaffold or fibers at the site of a bone defect. The bulk density of a cotton wool-like structure of the scaffold or fibers is about 0.01 to 0.2 g/cm3, preferably about 0.01 to 0.1 g/cm3, and the gaps between the fibers within the cotton wool-like structure are about 1-1000 μm, more preferably about 1-100 μm, such that body fluids can permeate throughout the gaps between the fibers and space for bone formation is ensured throughout the cotton wool-like structure.

In accordance with an embodiment of the invention, lengths of the biodegradable fibers are preferably about 5-20 mm, more preferably about 4-10 mm. According to an embodiment of the present invention, spinning solution produced by using kneading process contains a large amount of calcium particles such as 70 wt % or 43vo1%. When the spinning solution is ejected from the nozzle, calcium particles are bound each other by the polymer forming a long lengthwise fiber. However, when the trajectory of the ejected fiber is violently swung during the flight due to repulsion force in the electric field, the fiber formed of calcium particles and binder polymer can no longer maintain its lengthwise shape and are torn off to make shorter fibers.

After implantation of a scaffold or fibers at a bone defect site, body fluids containing mesenchymal stem cells may come into contact with the BMP-2 (e.g., rhBMP-2 or tBMP-2) captured on the β-TCP particles. The BMP-2 then promotes osteoprogenitor cells to differentiate into osteoblasts. β-TCP particles that bind BMP-2 are gradually dissolved by osteoclasts or other biological active components. Then, the osteoblasts work to form new bone on the β-TCP particles as in a bone remodeling process.

After implantation of a scaffold or fibers at a bone defect site, PLGA polymers in the electrospun fibers are gradually degraded such that β-TCP particles buried in the fibers would become exposed, and the newly exposed β-TCP particles would recapture the BMP-2 that were adhered on the surface of the fibers. As the degradation of PLGA proceeds, due to the recapture of BMP-2 by the newly exposed β-TCP particles, remodeling of bone continuously occurs throughout the network of the scaffold of biodegradable fibers, resulting in efficient bone formation at the bone defect site.

In accordance with embodiments of the invention, the BMP-2 (e.g., rhBMP-2 or tBMP-2) binds to the β-TCP and/or SiV particles exposed on the surfaces of the fibers such that the BMP-2 is captured onto the fibers throughout the cotton-wool like structure. The fusion of the β-TCP-binding peptide may be to the N- or C-terminus of the BMP-2.

The tBMP-2 can be produced with conventional molecular biological techniques or other techniques known in the art (such as chemical or enzymatic couplings of the (3-TCP-binding peptides to the BMPs). For example, the nucleic acid sequence for a 13-TCP-binding peptide may be attached to the nucleic acid sequence of the BMP using polymerase chain reactions (PCR). Alternatively, the fusion protein nucleic acid construct may be chemically synthesized. The fusion protein construct is then placed into an appropriate expression vector at the restriction sites. The expression vector is then transfected into a protein expression system (e.g., E. coli, yeast cells, or CHO cells). The expressed proteins are then purified. To facilitate protein purification, a specific tag (e.g., a histidine tag) may be constructed into the expression construct. All these processes and techniques are conventional and routine. One skilled in the art would be perform these without undue experimentation.

In accordance with embodiments of the invention, a β-TCP binding peptide may comprise the amino acid sequence LLADTTHHRPWT (SEQ ID NO: 1), GQVLPTTTPSSP (SEQ ID NO: 2), VPQHPYPVPSHK (SEQ ID NO: 3), HNMA-PATLHPLP (SEQ ID NO: 4), QSFASLTNPRVL (SEQ ID NO: 5), HTTPTTTYAAPP (SEQ ID NO: 6), QYGVVSHLTHTP (SEQ ID NO: 7), TMSNPITSLISV (SEQ ID NO: 8), IGRISTHAPLHP (SEQ ID NO: 9), MNDPSPWLRSPR (SEQ ID NO: 10), QSLGSMFQEGHR (SEQ ID NO: 11), KPLFTRYGDVAI (SEQ ID NO: 12), MPF-GARILSLPN (SEQ ID NO: 13), QLQLSNSMSSLS (SEQ ID NO: 14), TMNM-PAKIFAAM (SEQ ID NO: 15), EPTKEYTTSYHR (SEQ ID NO: 16), DLNE-LYLRSLRA (SEQ ID NO: 17), DYDSTHGAVFRL (SEQ ID NO: 18), SKHERYPQSPEM (SEQ ID NO: 19), HTHSSDGSLLGN (SEQ ID NO: 20), NYDSMSEPRSHG (SEQ ID NO: 21), or ANPIISVQTAMD (SEQ ID NO: 22), or a combination thereof, which are disclosed in U.S. Pat. No. 10,329,327 B2, the disclosure of which is incorporated by reference in its entirety. In some embodiments, alβ-TCP binding peptide comprises VIGESTHHRPWS (SEQ ID NO: 23), IIGESSHHKPFT (SEQ ID NO: 24), GLGDTTHHRPWG (SEQ ID NO: 25), or ILAESTHHKPWT (SEQ ID NO: 26), or a combination thereof. In some embodiments, a 13-TCP binding peptide comprises LLADTTHHRPWT (SEQ ID NO: 1), VIGESTHHRPWS (SEQ ID NO: 23), IIGESSHHKPFT (SEQ ID NO: 24), GLGDTTHHRPWG (SEQ ID NO: 25), and ILAESTHHKPWT (SEQ ID NO: 26).

In accordance with some embodiments of the invention, a β-TCP binding peptide may comprise two or more sequences selected from the above sequences. The two or more sequences may be directly connected to each other, or with a short peptide linker interspersed therebetween, to form a longer β-TCP binding peptide. Non-limiting example 13-TCP binding peptides include LLADTTHHRPWTVIGESTHHRPWSI-IGESSHHKPFTGLGDTTHHRPWGILAESTHHKPWT (SEQ ID NO: 27), LLADT-THHRPWTVIGESTHHRPWSIIGESSHHKPFTGLGDTTHHRPWG (SEQ ID NO: 28), LLADTTHHRPWTVIGESTHHRPWSIIGESSHHKPFT (SEQ ID NO: 29), LLADTTHHRPWTVIGESTHHRPWS (SEQ ID NO: 30), and VIGESTHHRPWSI-IGESSHHKPFTGLGDTTHHRPWGILAESTHHKPWT (SEQ ID NO: 31). Further example β-TCP binding peptides include a first peptide and a second peptide, wherein: the first peptide comprises SEQ ID NO: 1 and the second peptide comprises one or more of SEQ ID NOS: 23, 24, 25, or 26; the first peptide comprises SEQ ID NO: 23 and the second peptide comprises one or more of SEQ ID NOS: 1, 24, 25, or 26; the first peptide comprises SEQ ID NO: 24 and the second peptide comprises one or more of SEQ ID NOS: 23, 1, 25, or 26; the first peptide comprises SEQ ID NO: 25 and the second peptide comprises one or more of SEQ ID NOS: 23, 24, 1, or 26; the first peptide comprises SEQ ID NO: 26 and the second peptide comprises one or more of SEQ ID NOS: 23, 24, 25, or 1; the first peptide comprises SEQ ID NO: 1 and the second peptide comprises two or more of SEQ ID NOS: 23, 24, 25, or 26; the first peptide comprises SEQ ID NO: 23 and the second peptide comprises two or more of SEQ ID NOS: 1, 24, 25, or 26; the first peptide comprises SEQ ID NO: 24 and the second peptide comprises two or more of SEQ ID NOS: 23, 1, 25, or 26; the first peptide comprises SEQ ID NO: 25 and the second peptide comprises two or more of SEQ ID NOS: 23, 24, 1, or 26; the first peptide comprises SEQ ID NO: 26 and the second peptide comprises two or more of SEQ ID NOS: 23, 24, 25, or 1; the first peptide comprises SEQ ID NO: 1 and the second peptide comprises three or more of SEQ ID NOS: 23, 24, 25, or 26; the first peptide comprises SEQ ID NO: 23 and the second peptide comprises three or more of SEQ ID NOS: 1, 24, 25, or 26; the first peptide comprises SEQ ID NO: 24 and the second peptide comprises three or more of SEQ ID NOS: 23, 1, 25, or 26; the first peptide comprises SEQ ID NO: 25 and the second peptide comprises three or more of SEQ ID NOS: 23, 24, 1, or 26; or the first peptide comprises SEQ ID NO: 26 and the second peptide comprises three or more of SEQ ID NOS: 23, 24, 25, or 1.

Due to the presence of β-TCP-binding peptides, the bindings of tBMP-2 to the β-TCP particles are very tight, thereby further preventing tBMP-2 from leaking to outside of bone defect areas. As a result, safety of using the tBMP-2/fibers herein is further ensured.

ReBOSSIS

ReBOSSIS is a bone-void-filling material having cotton wool-like structures formed of biodegradable fibers. Details of ReBOSSIS are explained in U.S. Pat. Nos. 8,853,298, 10,092,650, U.S. patent application publication No. 2016/0121024, and U.S. patent application publication No. 2018/0280569. Disclosures of these references are incorporated herein by reference in their entirety.

The diameters of the electrospun biodegradable fibers of ReBOSSIS may range from about 40-320 μm, preferably about 80-250 μm, and more preferably about 90-200 μm. In contrast, the diameters of conventional electrospun fibers are usually several tens or several hundred nanometers (nm). Orthorebirth obtained thicker electrospun fibers by sending electrospinning (ES) solution to a large diameter nozzle at a fast rate and spinning the fibers by dropping the fiber from the top of ES apparatus to the bottom. Diameter of electrospun fiber becomes further thicker as the amount of calcium compound particles is increased, eventually resulting in diameter of more than 60 μm. Because electrospinning is known for producing very thin nanofibers, the methods used by Orthorebirth for producing thicker fibers are unique. Details of the method of Orthorebirth is described in PCT/JP2019/036052 filed on Sep. 13, 2019. By producing this thick electrospun biodegradable fiber, it became possible for the inventors to include a large amount of calcium compound particles in the fiber such that the particles are exposed on the fiber. Also, the thicker fibers have the mechanical strength to maintain the shape of the fibers after implantation at the bone repair sites.

Biodegradable fibers of ReBOSSIS contain large amounts of calcium particles (e.g., β-TCP, or SiV, or β-TCP+SiV). Inclusion of such large amounts of calcium particles is achieved by using a kneading process. Briefly, a mixture for the biodegradable fiber and calcium particles is kneaded in a kneader with a strong force to produce a composite. The composite is then dissolved in a solvent (e.g., chloroform) to produce a spinning solution. Details of the kneading process is described in WO2017/188435 filed on Apr. 28, 2017.

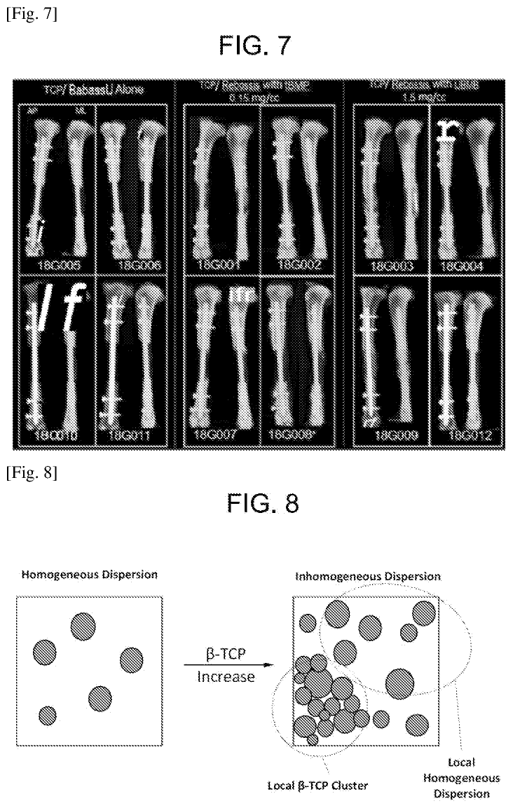

By adding calcium particles to the polymer in the kneader to knead the mixture of biodegradable polymer and calcium particles, calcium particles are homogeneously dispersed in the matrix polymer. However, if the volume ratio of calcium particles exceeds a threshold amount, due to the occurrence of percolation phenomenon, the particles can no longer maintain a homogeneous dispersion state, and cluster phase starts to appear (see FIG. 8). As a result of the cluster phase formation of the particles, some calcium particles are exposed on the surfaces of biodegradable fibers (see FIGS. 1A-1F). This makes it possible for BMP-2 to bind to the β-TCP particles on the biodegradable fibers. According to experience of inventors, this percolation phenomenon starts appearing when volume percentage of inorganic particles exceeds about 25 vol %. The relative vol % of calcium particles is calculated based on the total volume (100 vol %) of all components (i.e., biodegradable polymer and calcium compounds) in the biodegradable fibers. In an embodiment of the present invention, area of binding site of BMP-2 with β-TCP particles exposed on a surface of the electrospun biodegradable fibers is adjusted by an amount of the β-TCP particles contained in the electrospun biodegradable fibers.

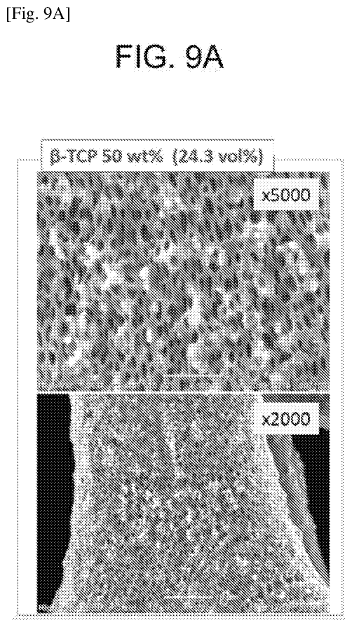

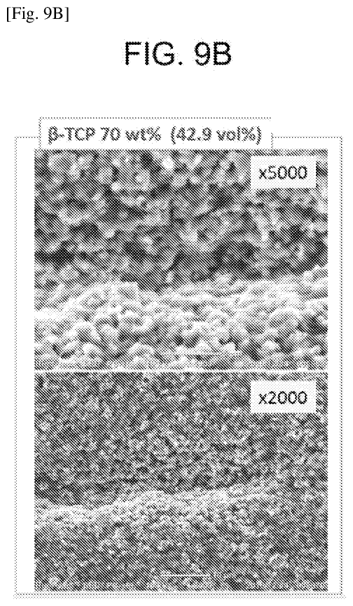

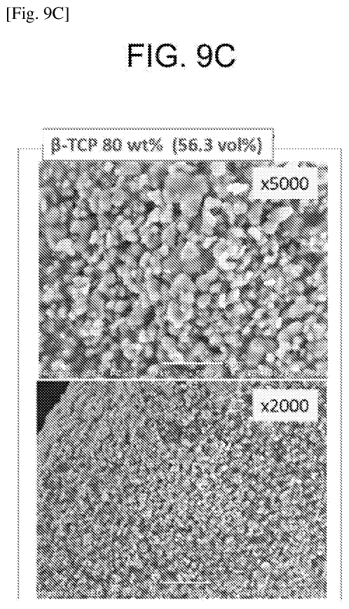

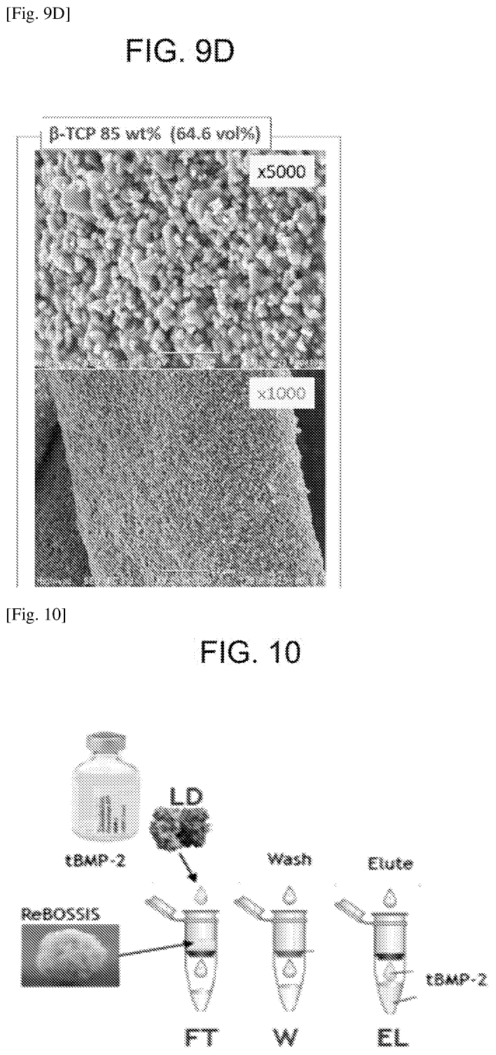

FIGS. 9A-9D show EM images of ReBOSSIS fibers of the invention, illustrating the exposures of β-TCP particles on the biodegradable fibers increasing as the vol % of 13-TCP particles contained in the biodegradable fibers increases. FIG. 9A shows the fibers with β-TCP (50 wt %, 24.3 vol %); there are not many β-TCP particles exposed on the surface of the fiber. FIG. 9B shows the fibers with β-TCP (70 wt %, 42.9 vol %); there are many β-TCP particles exposed on the surface of the fiber. FIG. 9C shows the fibers with β-TCP (80 wt %, 56.3 vol %); there are many more β-TCP particles exposed on the surface of the fiber. FIG. 9D shows the fibers with β-TCP (85 wt %, 64.6 vol %); there are even more β-TCP particles exposed on the surface of the fiber. Confirming that some β-TCP particles are exposed on the fibers Method.

A fibrous sheet having following composition was immersed into HCL of 1 mol/L for 10s, lmin, 5 min.
PLGA 30 wt %, β-TCP 70 wt %, or
PLGA 50 wt %, β-TCP 50 wt %

If the β-TCP particles are coated by polymer, the β-TCP particles will not be dissolved by HCl. Thus, there would be no changes. On the other hand, if the β-TCP particles are dissolved by HCl, it is assumed that the β-TCP particles are exposed and not being coated by the polymer.

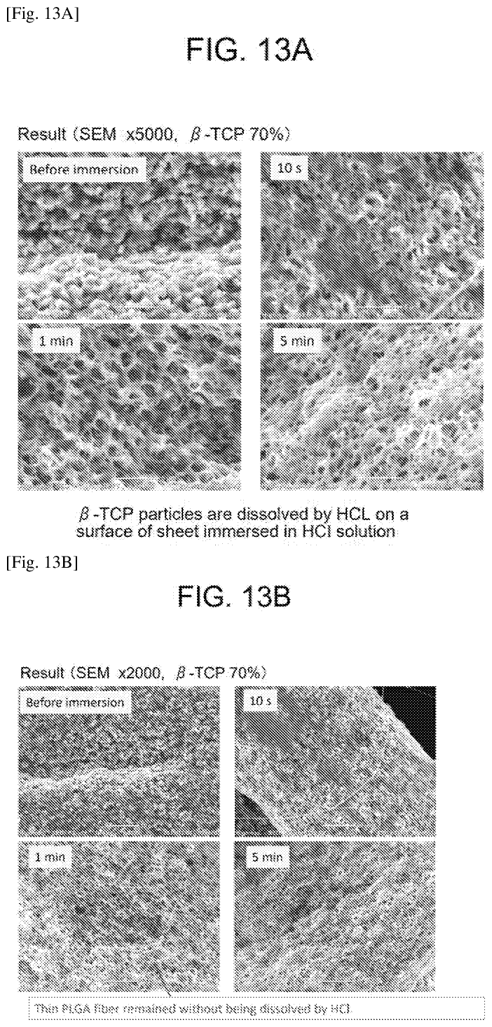

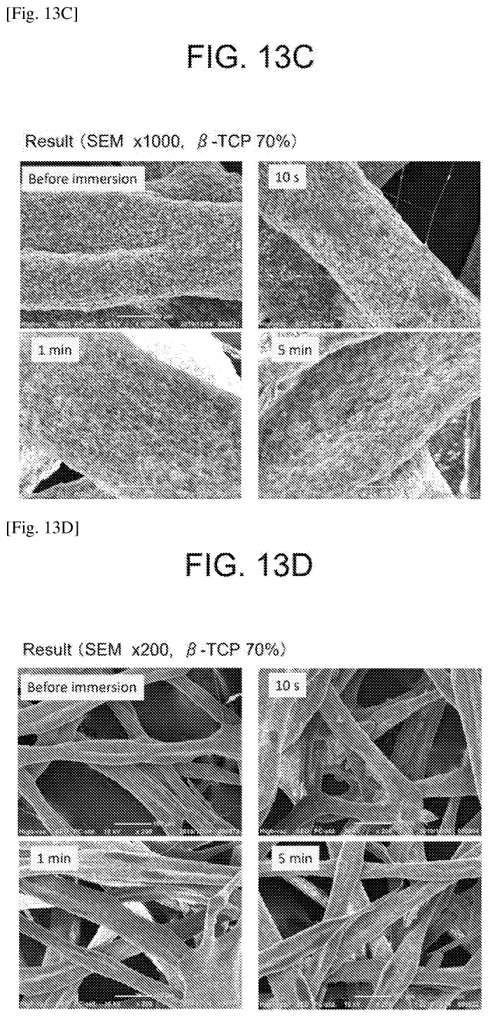

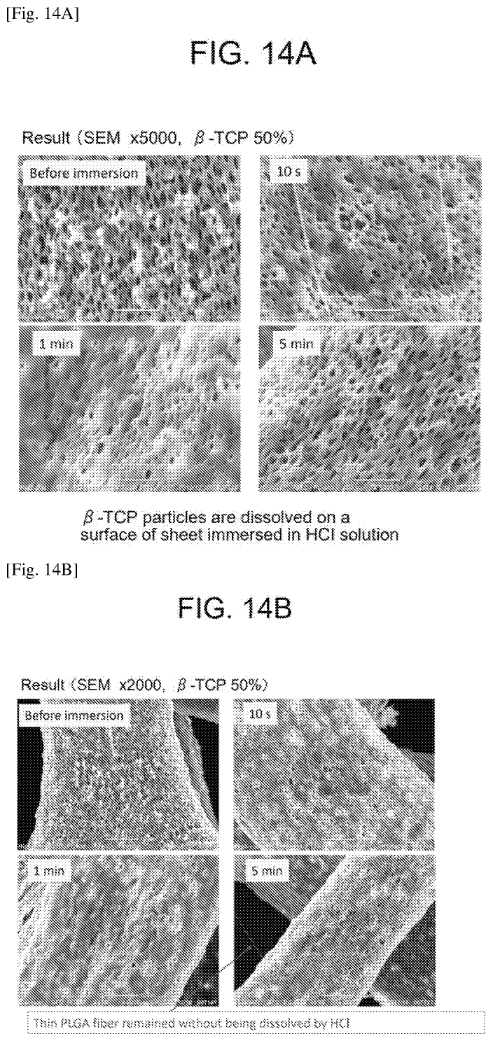

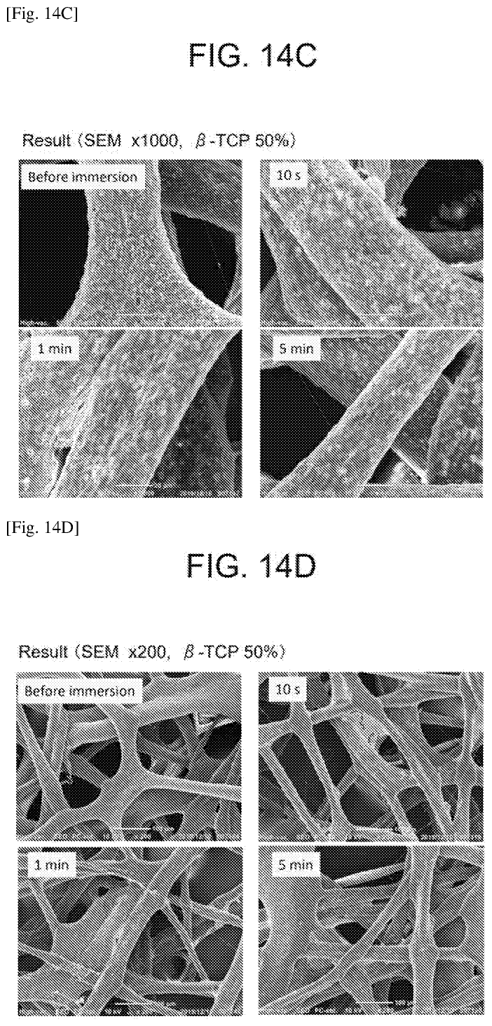

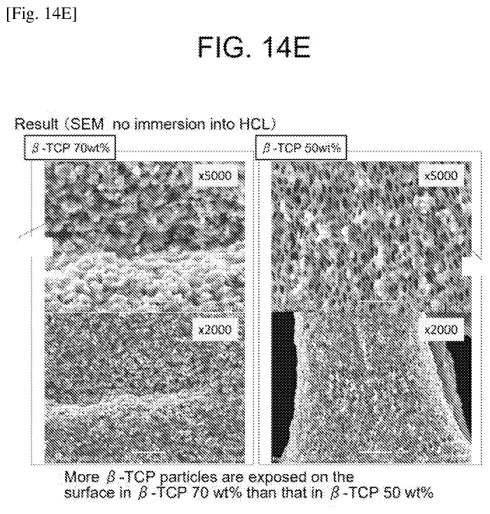

As shown in FIGS. 13A-13D, it was confirmed that β-TCP particles are dissolved by HCL. PLGA was not dissolved by HCl. The higher β-TCP ratio, more exposure of 13-TCP particles on the surface of the fibers. As revealed by these experiments, a portion of β-TCP particles in the fibers are not completely covered by the polymer. Therefore, HCl can dissolve these β-TCP particles. As shown in FIGS. 13A-13D and FIGS. 14A-14E, by changing the ratios of β-TCP particles to the polymers, the extents of exposure of β-TCP particles can be controlled. Accordingly, binding site of BMP-2 to the fibrous scaffolds can be controlled by increasing or decreasing the exposure of 13-TCP particles on the fibers.

tBMP-2

Some embodiments of the invention use targetable BMP-2 (tBMP-2), in which 13-TCP binding peptides are fused with BMP2. As a non-limiting example, BMP comprises QAKHKQRKRLKSSCKRHPLYVDFSDVGWNDWIVAPPGYHAFY-CHGECPFPLADHLNSTNHAIVQTLVNSVNSKIPKACCVPTELSAISMLYLDENE KVVLKNYQDMVVEGCGCR (SEQ ID NO: 32). tBMP-2 is fused with alβ-TCP binding peptide developed by one of the inventors of this invention. Details of certain β-TCP binding peptides are explained in U.S. Pat. No. 10,329,327 B2 and in “Tethering of Epidermal Growth Factor (EGF) to Beta Tricalcium Phosphate ((3TCP) via Fusion to a High Affinity, Multimeric β-TCP binding Peptide: Effects on Human Multipotent Stromal Cells/Connective Tissue Progenitors,” Alvarez et al., PLoS ONE DOI: 10.1371/journal.pone.0129600, Jun. 29, 2015. Disclosures of these references are incorporated herein by reference in their entirety.

In accordance with embodiments of the invention, a β-TCP binding peptide may comprise the amino acid sequence LLADTTHHRPWT (SEQ ID NO: 1), GQVLPTTTPSSP (SEQ ID NO: 2), VPQHPYPVPSHK (SEQ ID NO: 3), HNMA-PATLHPLP (SEQ ID NO: 4), QSFASLTNPRVL (SEQ ID NO: 5), HTTPTTTYAAPP (SEQ ID NO: 6), QYGVVSHLTHTP (SEQ ID NO: 7), TMSNPITSLISV (SEQ ID NO: 8), IGRISTHAPLHP (SEQ ID NO: 9), MNDPSPWLRSPR (SEQ ID NO: 10), QSLGSMFQEGHR (SEQ ID NO: 11), KPLFTRYGDVAI (SEQ ID NO: 12), MPF-GARILSLPN (SEQ ID NO: 13), QLQLSNSMSSLS (SEQ ID NO: 14), TMNM-PAKIFAAM (SEQ ID NO: 15), EPTKEYTTSYHR (SEQ ID NO: 16), DLNE-LYLRSLRA (SEQ ID NO: 17), DYDSTHGAVFRL (SEQ ID NO: 18), SKHERYPQSPEM (SEQ ID NO: 19), HTHSSDGSLLGN (SEQ ID NO: 20), NYDSMSEPRSHG (SEQ ID NO: 21), ANPIISVQTAMD (SEQ ID NO: 22), VIGESTHHRPWS (SEQ ID NO: 23), IIGESSHHKPFT (SEQ ID NO: 24), GLGDTTHHRPWG (SEQ ID NO: 25), or ILAESTHHKPWT (SEQ ID NO: 26), or a combination thereof.. In accordance with some embodiments of the invention, a β-TCP binding peptide may comprise two or more sequences selected from the above sequences. The β-TCP binding peptide may comprise LLADTTHHRPWT (SEQ ID NO: 1), VIGESTHHRPWS (SEQ ID NO: 23), IIGESSHHKPFT (SEQ ID NO: 24), GLGDTTHHRPWG (SEQ ID NO: 25), and ILAESTHHKPWT (SEQ ID NO: 26). The two or more sequences may be directly connected to each other, or with a short peptide interspersed therebetween, to form a longer β-TCP binding peptide.

In accordance with embodiments of the invention, the BMPs used in tBMPs may include a targetable BMP-2 and recombinant human BMP-2 (rhBMP-2). In some embodiments, provided is a tBMP comprising a β-TCP binding peptide (e.g., one or more β-TCP binding peptides disclosed herein) and QAKHKQRKRLKSSCKRHPLYVDFS-DVGWNDWIVAPPGYHAFYCHGECPFPLADHLNSTNHAIVQTLVNSVNSKIPK ACCVPTELSAISMLYLDENEKVVLKNYQDMVVEGCGCR. The β-TCP binding peptide may be connected to the BMP sequence via a linker, e.g., a peptide linker. Non-limiting examples of tBMP peptides include MPIGSLLADT-THHRPWTVIGESTHHRPWSIIGESSHHKPFTGLGDTTHHRPWGILAESTHHKPW TASGAGGSEGGGSEGGTSGATGAGTSTSGGGASTGGGTGQAKHKQRKRLKSS CKRHPLYVDFSDVGWNDWIVAPPGYHAFYCHGECPFPLADHLNSTNHAIVQT LVNSVNSKIPKACCVPTELSAISMLYLDENEKVVLKNYQDMVVEGCGCR (SEQ ID NO: 33), LLADTTHHRPWTVIGESTHHRPWSIIGESSHHKPFTGLGDT-THHRPWGILAESTHHKPWTASGAGGSEGGGSEGGTSGATGAGTSTSGGGAST GGGTGQAKHKQRKRLKSSCKRHPLYVDFSDVGWNDWIVAPPGYHAFYCHGE CPFPLADHLNSTNHAIVQTLVNSVNSKIPKACCVPTELSAISMLYLDENEKVVL KNYQDMVVEGCGCR (SEQ ID NO: 34), VIGESTHHRPWSIIGESSHHKPFT-GLGDTTHHRPWGILAESTHHKPWTASGAGGSEGGGSEGGTSGATGAGTSTSG GGASTGGGTGQAKHKQRKRLKSSCKRHPLYVDFSDVGWNDWIVAPPGYHAF YCHGECPFPLADHLNSTNHAIVQTLVNSVNSKIPKACCVPTELSAISMLYLDEN EKVVLKNYQDMVVEGCGCR (SEQ ID NO: 35), IIGESSHHKPFTGLGDT-THHRPWGILAESTHHKPWTASGAGGSEGGGSEGGTSGATGAGTSTSGGGAST GGGTGQAKHKQRKRLKSSCKRHPLYVDFSDVGWNDWIVAPPGYHAFYCHGE CPFPLADHLNSTNHAIVQTLVNSVNSKIPKACCVPTELSAISMLYLDENEKVVL KNYQDMVVEGCGCR (SEQ ID NO: 36), GLGDTTHHRPWGILAESTHHKPW-TASGAGGSEGGGSEGGTSGATGAGTSTSGGGASTGGGTGQAKHKQRKRLKSS CKRHPLYVDFSDVGWNDWIVAPPGYHAFYCHGECPFPLADHLNSTNHAIVQT LVNSVNSKIPKACCVPTELSAISMLYLDENEKVVLKNYQDMVVEGCGCR (SEQ ID NO: 37), and ILAESTHHKPWTASGAGGSEGGGSEGGTSGATGAGT-STSGGGASTGGGTGQAKHKQRKRLKSSCKRHPLYVDFSDVGWNDWIVAPPG YHAFYCHGECPFPLADHLNSTNHAIVQTLVNSVNSKIPKACCVPTELSAISMLY LDENEKVVLKNYQDMVVEGCGCR (SEQ ID NO: 38).

Binding BMP-2 to ReBOSSIS

In accordance with embodiments of the invention, BMP-2 can bind to the ReBOSSIS fibers. To evaluate the properties of BMP-2 binding to ReBOSSIS fibers, the following experiment was performed (using tBMP2 and rhBMP-2 as an example). In this experiment, biodegradable fibers of ReBOSSIS contains PLGA (30 wt %) and β-TCP particles (40 wt %) and SiV (silicon doped calcium carbonate of vaterite phase) particles (30 wt %). The β-TCP particles and SiV particles are distributed in and on the fibers. A portion of the particles is exposed outside on the surfaces of the fibers macro-scopically.

Four sample solutions were prepared. The concentrations of tBMP-2 or rhBMP-2 in the following four sample solutions were compared with a control sample using Poly-Acrylamide Gel Electrophoresis (SDS-PAGE). Bovine serum albumin (BSA) was used as the control sample.

Specifically, the following reagents were prepared: (a) tBMP-2 (SEQ ID NO: 33, in 10 mM Na-Acetate, 0.1M NaCl with or without 0.1 M Urea, pH 4.75); (b) BSA Stock solution: 42 mg/ml dissolved in Acetate Wash Buffer (store at −20° C.); (c) Acetate Wash Buffer: 5m M Na-acetate pH 4.75, 100 mM NaCl; (d) ReBOSSIS (OrthoRebirth); and (e) PBS (Roche, cat #11666789001, 1X solution=137 mM NaCl, 2.7 mM KCl, 10 mM Na2HPO4, 1.8 mM KH2PO4, pH 7.0).

Method: Bind 20 microgram tBMP-2 or BSA/mg ReBOSSIS (total 200 microgram tBMP-2 and 10 mg ReBOSSIS), wash, elute, and load onto Non-reducing SDS-PAGE. Specific protocols are as follows:

Preparation

1. Weigh 10 mg ReBOSSIS in spin tubes;

2. Prepare BSA: Take 29 microliter BSA Stock and add 971 microliter of acetate buffer to achieve a diluted BSA solution, 1.22 mg/ml at pH 4.75.

3. Prewash ReBOSSIS in acetate wash buffer by adding 500 microliter acetate wash buffer in each tube. Mix well end over end, let it incubate for 5 minutes. Spin down in a microfuge. To remove the buffer, place tip at bottom of tube and pipet. ReBOSSIS will remain in the tube.

4. Gel Sample: Set aside some BSA Load and tBMP2 load for later SDS-PAGE analysis.

Assay

5. Add 200 microgram tBMP2 to a 10 mg prewashed ReBOSSIS in a total volume of 164 microliter;

6. Add 200 microgram BSA to a 10 mg prewashed ReBOSSIS in a total volume of 164 microliter

TABLE 2
Example Setup

| Tube No. | BSA Control | tBMP-2 |
|---|---|---|
| BSA (1.22 mg/ml) | 164 | |
| tBMP2 (2.41 mg/ml) | — | 83 |
| Acetate pH 4.75 (100 mM NaCl) | — | 81 |

7. Bind for 30 minutes (binding is nearly complete at 20 minutes); Collect Unbound Material

8. Spin tubes for 4-5 minutes at the highest speed in a microfuge.

9. Insert a pipet into the supernatant at the top of the tube and pipet out the non-bound into a tube. (Note: Make sure not to get ReBOSSIS in your pipet tip. We take out excess amounts to run our gel. Then we place a tip in the bottom of the tube and discard the remainder); Wash

10. Add 500 microliter of either PBS or acetate wash buffer;

11. Gently Vortex. Mix end over end for 5 minutes, then gently vortex again;

12. Extract Wash by spinning tubes for 4-5 minutes at the highest speed in a microfuge. Keep the wash; Elute

13. Elute by adding 164 microliter non-reducing 1X SDS PAGE gel dye (Without (3-mercaptoethanol, as that will destroy the BMP2 dimer);

14. Vortex gently, incubate 5 minutes, repeat vortex;

15. Collect the eluted material by spinning tubes for 4-5 minutes at the highest speed in a microfuge;

16. Load gel as follows: ReBOSSIS Load:10 microliter, Non-Bound 10 microliter,

Wash 10 microliter, Elution 11 microliter.

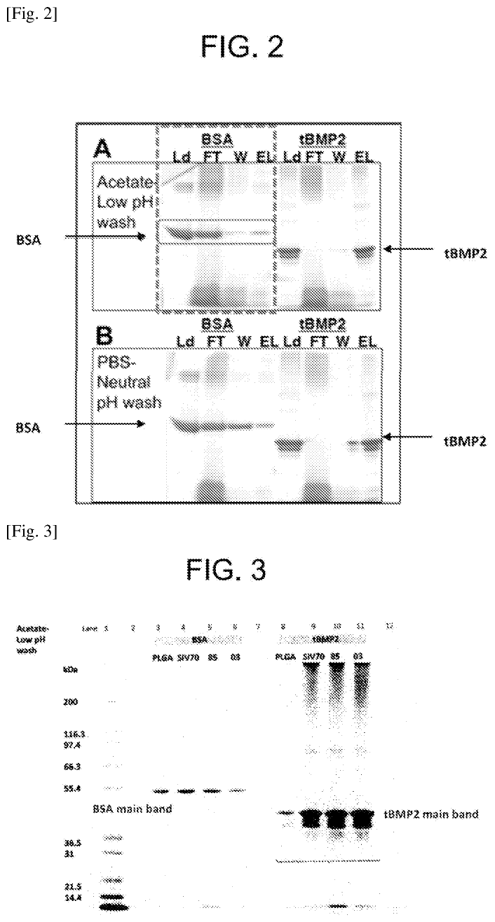

The samples for SDS PAGE analysis are labeled as follows (as shown in FIG. 2 and FIG. 10):

Ld: Loading sample solution containing a known amount of tBMP-2 or BSA.

FT: Flow-through sample solution obtained by collecting the fraction that flowed through ReBOSSIS after Ld was provided to ReBOSSIS.

W: Wash sample solution obtained by collecting the fraction that went through the protein-containing ReBOSSIS after a wash buffer was provided.

If protein that was bound to ReBOSSIS became separated by wash buffer, the fraction that flowed out after washing would contain the separated protein.

Assuming that the pH condition of the implant site is either acidic or neutral, two types of wash buffers (PBS pH 7.0 and acetate buffer pH 4.5) were prepared and used to conduct the experiments.

EL: Elution sample solution obtained by collecting the fraction after an elution buffer was provided to the ReBOSSIS after being washed by a wash buffer.

Evaluation of the binding of protein (SDS-PAGE)

The binding of tBMP-2 (or BSA) to ReBOSSIS was evaluated on SDS-PAGE, and a staining solution was used to detect the protein bands. The detected protein appears as a band in a lane. By comparing the signal intensity of an electrophoresis band of a sample having a known amount of protein with the signal intensity of an electrophoresis band of a sample with an unknown amount of protein, the unknown amount of protein can be quantitatively estimated. By using software for image analysis, the intensities of the signals can be quantitatively analyzed.

In this experiment, the gel image was compared by eye observations. A blue band shown in a lane was produced by staining the protein (tBMP2 or BSA) with a blue dye.

A denser band indicates a larger amount of the protein.

SDS-PAGE was conducted at a constant voltage of 130V. NuPAGE 4-12% Bis-Tris Protein Gels, 1.0 mm, 12-well (Life Technologies, cat #NP0322BOX) were used, and NuPAGE MOPS SDS Running Buffer (20X) (Life Technologies cat #NP0001) was used as the electrophoresis buffer. In order to stain the protein after electrophoresis is completed, Gelcode Blue Sage Protein Stain (Thermos Scientific, cat #24596) was used.

As shown in FIG. 2, Panel A shows a gel image obtained using an acidic buffer (acetate buffer) for wash buffer, and Panel B shows a gel image obtained using a neutral buffer (PBS) for wash buffer. In each gel image, the four right lanes show results of analysis of tBMP2, and the four left lanes show results of analysis of BSA.

In the Ld lanes (loading samples), the positions of main bands of tBMP-2 and BSA can be identified. If tBMP-2 or BSA is contained in the FT, W, or EL lane, its band is expected to appear at the same position as that in the Ld lane.

In FIG. 2, the area surrounded by dotted lines shows gel image of the sample that was prepared using BSA under condition A. The position of the main band of BSA is indicated by a rectangular solid line box, which is denoted as BSA main band. From a comparison of the intensities of the main bands of BSA in each lane, it is clear that the FT and Ld lanes show similar levels of proteins, indicating that most BSA does not bind ReBOSSIS and flow right through. That is, BSA acts as a negative control that does not bind ReBOSSIS fibers. Although the W and EL lanes also show trace amount of BSA bands, the levels of BSA bands in the W and EL lanes are at much lower levels, as compared with that of the Ld lane, indicating that little BSA was bound to ReBOSSIS.

In the neutral pH buffer (panel B), BSA show some non-specific binding to the ReBOSSIS fibers, however, most BSA came through in the flow through fraction (FL), indicating that most BSA does not bind the ReBOSSIS fibers. In the wash fraction (W), some BSA continues to come through, but the amount is less than the flow through faction. The amount is even less in the elution (EL) fraction. These results indicate some non-specific sticking of BSA to the ReBOSSIS fibers.

In contrast, tBMP-2 binds well to ReBOSSIS and very little came out in the flow through (FT) or the wash (W) fractions, either using an acidic buffer or a neutral pH buffer. The bound tBMP-2 came out only after elution (EL). (FIG. 2, Panel A and Panel B), indicating specific binding of tBMP-2 to the ReBOSSIS fibers.

From this experiment, it was confirmed that tBMP-2 can bind tightly to ReBOSSIS Fibers, and that tBMP-2 bound to ReBOSSIS fibers is not separated from ReBOSSIS fibers by acidic or neutral wash buffer.

From this experiment, it was confirmed that under both neutral and acidic conditions, greater than 98% of tBMP2 was bound and retained on ReBOSSIS. This means that even under the effect of osteoclast resorption, binding of tBMP-2 to ReBOSSIS continues.

Comparison of retention between tBMP-2 and rhBMP(Infuse)

This experiment is to compare the binding properties of tBMP-2 and rhBMP-2 (INFUSE) to ReBOSSIS fibers. The tests were conducted at several composition ratios as shown in the following table:

TABLE 3

| No. | Sample Name | Composite Material | PLGA | SiV | β-TCP |
|---|---|---|---|---|---|
| 1 | PLGA100 | Negative control | 100 wt % | — | — |
| 2 | SiV70 | SiV | 30 wt % | 70 wt % | — |
| 3 | ReBOSSIS (85) | SiV and β-TCP | 30 wt % | 30 wt % | 40 wt % |
| 4 | ORB-03 | β-TCP | 30 wt % | — | 70 wt % |

PLGA: poly(tactic-co-glycolic acid) PLA:PGA = 85:15 MW = 31000-380000
SiV: Siloxane-containing vaterite (a form of calcium carbonate, CaCO3)
β-TCP: β-Tricalcium phosphate

The binding protocols are as described above. FIG. 3 shows the results of bindings of tBMP-2 to the various materials. On the materials (SiV70, ReBOSSIS (85), and ORB-03) containing β-TCP and/or SiV (siloxane-containing vaterite), tBMP-2 is well retained. The retention of tBMP-2 is clearly different from that of BSA.

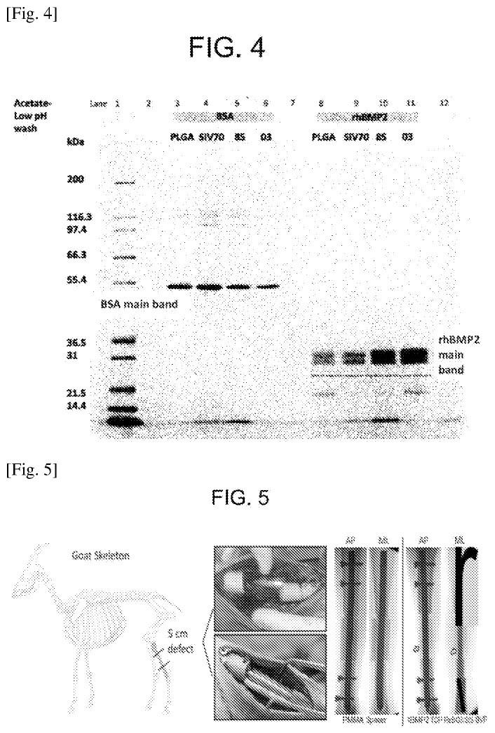

FIG. 4 shows results for recombinant human BMP2 (rhBMP2). rhBMP-2 is only retained on materials containing β-TCP (ReBOSSIS (85) and ORB-03), but not on material containing SiV. The binding of rhBMP-2 is weaker than the binding of tBMP2. Because retention of rhBMP-2 by ReBOSSIS is less than that of tBMP-2, one can predict that tBMP2 is less likely to leak out of the treatment site. Thus, preferred embodiments of the invention may use tBMP-2, which is expected to have fewer (if any) adverse effects, as compared with rhBMP-2 (e.g., INFUSE Bone Graft).

Evaluation of ReBOSSIS/tBMP2 in a Chronic Caprine Tibial Defect (CCTD) Model

To evaluate the utility of tBMP-2/ReBOSSIS in bone repair, the efficacy of targetable BMP-2 (tBMP-2 having SEQ ID NO: 33) on ReBOSSIS(85) was evaluated in the CCTD model, which is a challenging long-bone segmental defect goat model. It is expected that prolonged local retention of surface-bound tBMP2 at implantation sites improves the safety and efficacy of orthopedic procedures to correct long-bone segmental defects compared to the current practice.

Study Design

Animal Selection

Twelve (12) female Spanish Boer goats weighing between 40-60 kg were used for the study. They were divided into the following three experimental groups:

Group 1 TCP+ReBOSSIS+BMA alone

Group 2 TCP+ReBOSSIS+BMA+tBMP-2 @0.15 mg/cc defect

Group 3 TCP+ReBOSSIS+BMA+tBMP-2 @1.5 mg/cc defect TCP, tricalcium phosphate granules; BMA, bone marrow aspirate

CCTD Model

The CCTD model is intended to “raise the bar” for large animal models and better match the challenging clinical biological settings where current treatments for large bone defects continue to fail with unacceptable frequency.

The CCTD model involves a critical size (5 cm) segmental tibial defect of bone. Several features distinguish the CCTD model from acute defect models:

1.2 cm of periosteum is removed from each end of the defect site, creating a 9 cm segment of periosteum (the 5-cm defect+2 cm on either side),

2.10 grams of skeletal muscle around the defect site,

3.the intramedullary canal is reamed removing marrow and endosteal bone adjacent to the defect site, and

4.a PMMA spacer is placed in the defect for 4 weeks prior to grafting. This allows the spacer to be enveloped by a fibrous “induced membrane” (IM) or “Masquelet membrane.”

5.Each animal undergoes two surgeries defined here as the “Pre-procedure” to create these biological conditions and the “Treatment Procedure,” in which clinically relevant treatment scenarios can be implemented.

FIG. 5 shows a Schematic of Chronic Caprine Critical Defect (CCTD) Model. A 5-cm segment of critical defect is created in skeletally mature female goats during the pre-procedure. A 5-cm long ×2 cm diameter polymethylmethacrylate (PMMA) spacer is placed in the defect to induce a biological membrane. Four weeks later, the PMMA spacer is gently removed and replaced with the grafting materials. Orthogonal radiographs are taken every four weeks to assess defect healing. In the figure, AP represents craniocaudal, and ML represents mediolateral. White arrows indicate grafting material in placement of PMMA spacers.

The Pre-Procedure comprises the following essential features:

1.Creation of a craniomedial skin incision and excision of a 5-cm segment of tibial diaphysis and periosteum.

2.Excision of an additional 2 cm of periosteum on the proximal and distal bone segments.

3.Debridement of 10 cm3 of tibialis anterior and gastrocnemius muscles.

4.Placement of interlocking intramedullary nail with custom spacer clamp to maintain 5 cm defect.

5.Placement of a pre-molded 5 cm long ×2 cm diameter PMMA spacer around the nail in the defect.

6.Irrigation of the wound normal (0.9%) saline and wound closure.

The Treatment Procedure to be performed 4 weeks after the Pre-Procedure comprises:

1.Opening the previous skin incision on the craniomedial aspect of the tibia.

2.Opening the “induced membrane” around the PMMA spacer using a “bomb bay door opening.”

3.Spacer removal without damaging the membrane or nail.

4.Collection of appropriate tissue samples as defined below.

5.Placement of appropriate treatment into the defect.

6.Closure of the induced membrane with 3-0 nylon to provide an intrinsic marker and wound closure.

Radiographic Analysis:

Fluoroscopic imaging of the tibiae, anteroposterior (AP) and mediolateral (ML) projections were performed after the spacer procedure (week 0), the graft procedure (week 4), and follow-ups (week 8 and week 12). Radiographs were obtained after euthanasia (after soft tissue were dissected) 12 weeks after the grafting procedure.

Sample Preparation

Sample compositions: 5 cc TCP+50 cc ReBOSSIS+6 cc BMA (with or without tBMP-2).

Binding tBMP2 to ReBOSSIS

1. In a sterile environment, tease out 50cc ReBOSSIS in a petri-dish making sure it is spread out in a substantially uniform layer;

2.Add 30 mL binding buffer (with or without tBMP-2) to ReBOSSIS, by gently pipetting over the exposed surface and submerge ReBOSSIS in the solution, bind for 20 min;

3.Take out 30 mL binding buffer using a 10 mL pipette, by holding it vertically and pushing the tip to the surface of the place. While holding it to the surface suck up liquid carefully. Move the pipette to other areas to make sure to collect as much liquid as possible. Monitor the recovery volume;

4.Add 40 mL sterile PBS onto ReBOSSIS to wash for 10 min. Add the same way as described in step 2;

5.Take out the 40 mL PBS using a 10 mL pipette as described in step 3, store PBS in a 50 mL conical tube;

6.Repeat 1×step 4-5;

7.Put the lid on the Petri dish and Parafilm the edge to keep the tBMP2/ReBOSSIS sealed.

Binding tBMP2 to TCP

1.Measure the desired amount of TCP and place into a sterile tube.

2.Sterilize TCP by filling the tube with 70% Ethanol and incubating for 2-4 hours or overnight.

3.Wash TCP with sterile double deionized Water 3×to remove alcohol.

4.Wash TCP for 5 minutes in TCP binding buffer (10 mM Sodium Acetate pH 4.75, 100 mM NaCl) while gently agitating.

5.Wash TCP with sterile PBS to remove the TCP binding buffer.

6.Add the appropriate amount of tBMP2 to the TCP in the tube.

7.Add TCP binding buffer sufficient to cover the TCP.

8.Mix gently for 2 hours.

9.Wash with PBS X2 to remove the TCP binding buffer.

10.Store tBMP-2/TCP in the sterile container at 4 degrees Celsius.

Time of Surgery

1.Open one dish containing tBMP2/ReBOSSIS.

2.Find a corner of the same dish, decant the 5 ccs of tBMP-2 coated TCP. Then apply the 6 cc of bone marrow aspirate to the TCP and distribute evenly.

3.Use a sterile spatula transfer tBMP-2/TCP/BMA on top of the ReBOSSIS, making sure it spreads out evenly across the entire pile of ReBOSSIS.

4.Use sterile gloved hand gently pad the abovementioned tBMP-2/TCP/BMA so it's evenly distributed on the ReBOSSIS like a layer of fine pebbles.

5.Gently roll ReBOSSIS up like a burrito and mix and shape as needed Results

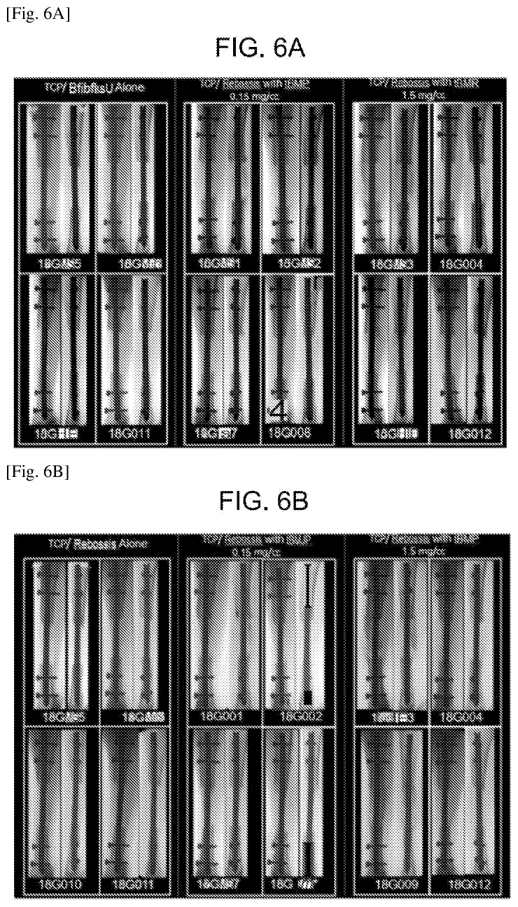

The surgical handling property of grafting materials was greatly enhanced by the addition of ReBOSSIS. The tBMP-2-containing groups (Group 2 and 3) showed higher percentages of new bone formation compared to Group 1. FIG. 6A shows the radiographs (mediolateral (ML) and craniocaudal (AP) projections) taken 8 weeks after grafting surgery, and FIG. 6B shows the radiographs (mediolateral (ML) and craniocaudal (AP) projections) taken 12 weeks after grafting surgery. Six (6) goats were used per treatment group.

The post-explant x-rays showed that no new bone was obtained in any of the defect sites for all 4 goats in Group 1 (TCP+ReBOSSIS+BMA). In Group 2 (low dose tBMP2), one of 4 goats had about 75% of new bone growth filled in the defect site, the other 3 goats had less than 25% new bone filled in the defect sites. In Group 3 (higher dose tBMP-2), 2 goats presented bone union and 2 goats presented less than 50% new bone. These data indicate that the tBMP2 addition to the scaffold did increase new bone formation.

FIG. 7 shows radiographs (mediolateral (ML) and craniocaudal (AP) projections) of the 12 explanted tibias taken with a fixed x-ray machine. Large amount of new bone was obtained in the higher dose tBMP-2 group (1.5 mg/cc).

As shown in these results, ReBOSSIS greatly enhanced the surgical handling property of the implant material. The addition of tBMP2 to TCP and ReBOSSIS enhanced the bone healing in the CCTD model. These results indicate that embodiments of the invention would be superior to presently used materials for bone repair. While these particular examples use tBMP-2, rhBMP-2 would produce the same results as having been demonstrated before.

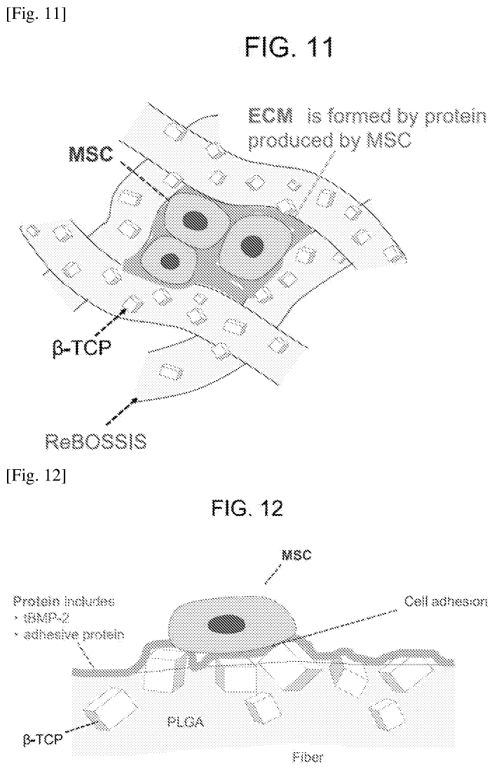

FIG. 11 and FIG. 12 show schematics illustrating possible processes for the enhanced bone repair using embodiments of the invention. Briefly, after implementation of ReBOSSIS, which had been dipped in a tBMP-2 solution, in a bone defect site, the surfaces of the fibers are likely coated by extracellular matrix (ECM) proteins or adhesive proteins present in the body fluids. Mesenchymal stem cells (MSCs) in the bone microenvironment then adhere to the surfaces of the fibers that are coated by adhesive proteins. MSCs may also produce proteins that form its own ECM. tBMP-2 induces MSCs differentiate into osteoblasts, which then form bone in the calcium rich environment.

While embodiments of the invention have been illustrated with a limited number of examples. One skilled in the art would appreciate that other modifications and variations are possible without departing from the scope of the invention. Therefore, the scope of the protection should only be limited with the attached claims.

1. A method of producing an osteoinductive bone regeneration material formed of a plurality of electrospun biodegradable fibers, comprising:
preparing a fibrous scaffold material formed of the plurality of electrospun biodegradable fibers, wherein the plurality of electrospun biodegradable fibers are 40-320 μm in diameter and 5-20 mm in length, wherein
the plurality of electrospun biodegradable fibers entangle with each other to form a cotton-wool like structure having inter-fiber spaces forming a microenvironment for cell growth therein,
the electrospun biodegradable fibers comprise 43-60 vol % β-TCP particles distributed in the electrospun biodegradable fibers such that a portion of the β-TCP particles is partially exposed on a surface of the electrospun biodegradable fibers without being coated by a polymer layer, and
immersing the fibrous scaffold in a solution containing BMP-2 so that the BMP-2 is bound to the β-TCP particles exposed on the surface of the fibers forming the microenvironment throughout the cotton-wool like structure to produce the osteoinductive bone graft, wherein an area of binding site for BMP-2 on β-TCP particles exposed on a surface of the electrospun biodegradable fibers is adjusted by an amount of the β-TCP particles contained in the electrospun biodegradable fibers.

2. The method of claim 1, wherein diameters of the plurality of electrospun biodegradable fibers are 70-250 μm.

3. The method of claim 1, wherein lengths of the plurality of electrospun biodegradable fibers are 4-10 mm.

4. The method of claim 1, wherein the plurality of electrospun biodegradable fibers comprise PLGA.

5. The method of claim 1, wherein diameters of the β-TCP particles are 2-5 μm.

6. The method of claim 1, wherein the BMP-2 is targetable BMP-2.

7. An osteoinductive bone regeneration material produced by the method of claims 1-6.

8. A fibrous scaffold material for osteoinductive bone regeneration material comprising a plurality of electrospun biodegradable fibers, wherein the plurality of electrospun biodegradable fibers is 40-320 μm in diameter and 5-20 mm in length,
wherein the plurality of electrospun biodegradable fibers are entangled with each other to form a cotton-wool like structure having inter-fiber spaces forming a microenvironment for cell growth therein,
wherein the electrospun biodegradable fibers comprise 45-60 vol % 13-TCP particles distributed in the electrospun biodegradable fibers such that a portion of the β-TCP particles is partially exposed on a surface of the electrospun biodegradable fibers without being coated by a polymer layer,
wherein an area of binding site for BMP-2 on β-TCP particles exposed on a surface of the electrospun biodegradable fibers is adjusted by an amount of the β-TCP particles contained in the electrospun biodegradable fibers.

9. The fibrous scaffold material of claim 8, wherein diameters of the plurality of electrospun biodegradable fiber are 70-250 μm.

10. The fibrous scaffold material of claim 8, wherein lengths of the plurality of electrospun biodegradable fibers are 4-10 mm.

11. The fibrous scaffold material of claim 8, wherein the electrospun biodegradable fibers comprise PLGA.

12. The fibrous scaffold material of claim 8, wherein diameters of the β-TCP particles are 2-5 μm.

13. The fibrous scaffold material of claim 8, wherein the β-TCP particles are bound to BMP-2.

14. The fibrous scaffold material of claim 13, wherein the BMP-2 is targetable BMP-2.

15. The fibrous scaffold material of any one of the previous claims, wherein the BMP-2 comprises any one of SEQ ID NOS: 1-38, or a combination or two more sequences from SEQ ID NOS: 1-38.

16. A composition comprising:
a scaffold comprising about 60 wt % to about 80 wt % calcium containing compound, and
a targetable BMP-2 comprising (i) VIGESTHHRPWS (SEQ ID NO: 23, (ii) IIGESSHHKPFT (SEQ ID NO: 24), (iii) GLGDTTHHRPWG (SEQ ID NO: 25), (iv) ILAESTHHKPWT (SEQ ID NO: 26), or (v) a combination of two more of (i)-(iv).

17. The composition of claim 16, wherein the targetable BMP-2 comprises VIGESTHHRPWS (SEQ ID NO: 23).

18. The composition of claim 16 or claim 17, wherein the targetable BMP-2 comprises IIGESSHHKPFT (SEQ ID NO: 24).

19. The composition of any one of claims 16-18, wherein the targetable BMP-2 comprises GLGDTTHHRPWG (SEQ ID NO: 25).

20. The composition of any one of claims 16-19, wherein the targetable BMP-2 comprises ILAESTHHKPWT (SEQ ID NO: 26).

21. The composition of any one of claims 16-20, wherein the targetable BMP-2 further comprises LLADTTHHRPWT (SEQ ID NO: 1).

22. The composition of any one of claims 16-21, wherein the targetable BMP-2 comprises QAKHKQRKRLKSSCKRHPLYVDFSDVGWND-WIVAPPGYHAFYCHGECPFPLADHLNSTNHAIVQTLVNSVNSKI PKACCVPTELSAISMLYLDENEKVVLKNYQDMVVEGCGCR (SEQ ID NO: 32).

23. The composition of any one of claims 16-22, wherein the targetable BMP-2 comprises any one of SEQ ID NOS: 33-38.

24. The composition of any one of claims 16-22, wherein the targetable BMP-2 comprises SEQ ID NO: 33.

25. The composition of any one of claims 16-24, wherein the calcium containing compound comprises calcium phosphate, vaterite, or calcium phosphate and vaterite.

26. The composition of any one of claims 16-24, wherein the calcium containing compound comprises beta-tricalcium phosphate (β-TCP).

27. The composition of claim 26, wherein the β-TCP is present in the scaffold at about 60 wt % to about 80 wt % of the scaffold.

28. The composition of claim 27, wherein the β-TCP is present in the scaffold at about 70 wt %.

29. The composition of claim 26, wherein the β-TCP is present in the scaffold at about 30 wt % to about 50 wt % of the scaffold.

30. The composition of claim 29, wherein the β-TCP is present in the scaffold at about 40 wt %.

31. The composition of any one of claims 16-26 or claims 29-30, wherein the calcium containing compound comprises vaterite.

32. The composition of claim 31, wherein the vaterite is present in the scaffold at about 20 wt % to 40 wt % of the scaffold.

33. The composition of claim 32, wherein the vaterite is present in the scaffold at about 30 wt %.

34. The composition of any one of claim 25 or 31-33, wherein the vaterite comprises silicon-doped vaterite (SiV).

35. The composition of any one of claims 16-34, wherein the scaffold comprises a biodegradable polymer.

36. The composition of any one of claims 16-34, wherein the scaffold comprises poly(lactic-co-glycolic acid) (PLGA).

37. The composition of claim 36, wherein the scaffold comprises about 20 wt % to about 40 wt % PLGA.

38. The composition of claim 37, wherein the scaffold comprises about 30 wt % PLGA.

39. A method of treating a subject in need thereof, comprising admin-istering to the subject a composition of any previous claim.

40. The method of claim 38, wherein the subject has a bone defect.
